(19) United States
(12) Patent Application Publication
(10) Pub. No.: US 2023/0057208 A1
(43) Pub. Date: Feb. 23, 2023

(54) DEVICES, SYSTEMS, AND METHODS FOR ACTIVATION OF A PHOTOACTIVE AGENT

(71) Applicant: BOSTON SCIENTIFIC SCIMED, INC., Maple Grove, MN (US)

(72) Inventors: Conor O'Sullivan, Cork (IE); Stephen Nolan, Cork (IE); John Murphy, Cork (IE); Lauren Sfakis Lydecker, Millbury, MA (US); Edward Joseph Devlin, Cork (IE); Barbara Santry, Cork (IE); Frank Ryan, Cork (IE)

(73) Assignee: BOSTON SCIENTIFIC SCIMED, INC., Maple Grove, MN (US)

(21) Appl. No.: 17/889,329

(22) Filed: Aug. 16, 2022

Related U.S. Application Data
(60) Provisional application No. 63/233,866, filed on Aug. 17, 2021.

(51) Int. Cl. A61N 5/06

(52) U.S. Cl. CPC A61N 5/062; A61N 5/0603; A61N 2005/061; A61N 2005/0629; A61N 2005/0628; A61N 2005/0652; A61N 2005/0658

(57) ABSTRACT

This disclosure relates generally to medical devices, systems, and methods for activation of a photoactive agent during a medical procedure. In an aspect, a catheter system for activation of a photoactive agent may comprise a catheter comprising an elongate shaft comprising a proximal portion, a distal portion, and a first lumen extending along the elongate shaft. A first flexible circuit may be disposed about the distal portion of the elongate shaft. A plurality of first light-emitting diodes (LEDs) may be disposed along the first flexible circuit. A second flexible circuit may be disposed about the distal portion of the elongate shaft. A plurality of second LEDs may be disposed along the second flexible circuit. A control unit may be coupled to the proximal end of the elongate shaft. The communications cable may be disposed within the catheter and may electrically couple the first LEDs with the control unit.

### CROSS-REFERENCE TO RELATED APPLICATIONS

This application claims the benefit of U.S. Provisional Patent Application Ser. No. 63/233,866, filed Aug. 17, 2021, which is incorporated herein by reference.

### FIELD

This disclosure relates generally to medical devices, systems, and methods for activation of a photoactive agent during a medical procedure.

### BACKGROUND

Photodynamic therapy (PDT) is a form of phototherapy involving a photosensitizing chemical agent activatable by light to cause tissue disruption. For example, a patient may be provided with a photoactive agent that is absorbed by cancerous cells. The therapy may include positioning light radiation near the absorbed cancerous cells, activating the photoactive agent, and disrupting the cancerous cells. PDT may have advantages over more invasive procedures such as decreased recovery time. However, PDT may risk damaging non-target tissue, e.g., adjacent the cancerous cells.

Light radiation catheters may require cumbersome capital equipment and allow for limited maneuverability. Available capital equipment for a procedure may provide a range of light having too broad or too specific of wavelengths that limit therapy effectiveness. Such hardships may discourage PDT, reducing service availability for patients. It is with these considerations that the devices, systems, and methods for activation of a photoactive agent of this disclosure may be useful.

### SUMMARY

According to an aspect, a catheter system for activation of a photoactive agent may comprise a catheter comprising an elongate shaft comprising a proximal portion, a distal portion, and a first lumen extending along the elongate shaft. A first flexible circuit may be disposed about the distal portion of the elongate shaft. A plurality of first light-emitting diodes (LEDs) may be disposed along the first flexible circuit. A second flexible circuit may be disposed about the distal portion of the elongate shaft. A plurality of second LEDs may be disposed along the second flexible circuit. A control unit may be coupled to the proximal end of the elongate shaft. A communications cable may be disposed within the catheter and electrically coupling the first and second LEDs with the control unit. The control unit may comprise a battery power supply and a controller comprising an integrated circuit electrically coupled to the plurality of first and second LEDs configured to vary power provided to the plurality of first and second LEDs. The controller may be configured to vary power provided to the plurality of second LEDs independently of the first LEDs.

In various embodiments described herein and otherwise within the scope of the present disclosure, the proximal portion of the elongate shaft may comprise the first lumen. The first flexible circuit and the second flexible circuit may be disposed about the distal end of the elongate shaft in a helical manner. A cross-section of the catheter system taken normal to the first lumen at an LED of the first LEDS may be coincident with an LED of the second LEDs. The controller may be configured to sequentially vary power between the plurality of first LEDs and the plurality of second LEDs. The controller may be configured to sequentially vary power to a frequency of about 45 Hz. The first LEDs may be configured to emit light at a first wavelength and the second LEDS are configured to emit light at a second wavelength different than the first wavelength. The first LEDs may be configured to emit ultraviolet or visible light. At least one LED of the first LEDs may be configured to emit a frequency of light different than a frequency of light emitted by another LED of the first LEDs. A photodiode disposed on the first flexible circuit may be configured to detect light emitted from the first LEDs. A second lumen may extend through the proximal portion and the distal portion of the elongate shaft. The second lumen may be configured to accept at least one of a guidewire, the photoactive agent, and a fluid to be delivered into a patient. The first lumen may extend along the proximal portion of the elongate shaft but not the distal portion of the elongate shaft. A connector may be disposed on the control unit configured to connect the battery power supply to a primary power supply for charging the battery.

In an aspect, a catheter system for activation of a photoactive agent may comprise a catheter for activation of a photoactive agent comprising an elongate shaft. The elongate shaft may comprise a proximal end, a distal end, and a first lumen therethrough. A plurality of circuits may be disposed helically about the distal end of the elongate shaft. A plurality of light-emitting diodes (LEDs) may be disposed along each of the plurality of circuits. A communications cable may be disposed within the first catheter lumen and may be electrically coupled independently to each of the plurality of circuits such that each of the plurality of circuits are independently activatable.

In various embodiments described herein and otherwise within the scope of the present disclosure, a cross-section of the catheter taken normal to the lumen at an LED of the plurality of LEDS may be coincident with an LED of each of the plurality of circuits. The LEDs of one circuit of the plurality of circuits may be configured to emit light at a wavelength different than a wavelength of the LEDs of the remaining plurality of circuits. A photodiode may be disposed on one of the plurality of circuits configured to detect light emitted from the plurality of LEDs. The communications cable may be configured to sequentially transfer power independently among each of the plurality of circuits.

In an aspect, a method of activating a photoactive agent may include introducing the photoactive agent into a tissue of a patient. A catheter comprising a first plurality of light-emitting diodes (LEDs) and a second plurality LEDs may be inserted into the patient towards the tissue. The photoactive agent may be illuminated by sequentially varying a power supplied to the first plurality of LEDs and the second plurality of LEDs. Tissue temperature may be monitored. The power supplied may be varied based on the monitoring. The photoactive agent may be locally introduced into the tissue. The photoactive agent may be intravenously introduced into the patient. The tissue may be selected from bladder tissue, pancreatic tissue, esophageal tissue, and lung tissue. A light emitted from either of the first and second plurality of LEDs may be sensed via a photo diode. The photoactive agent may comprise one of an anti-cancer compound and a photocurable or a photocrosslinkable agent. The photoactive agent may comprise tetra(hydroxyphenyl)chlorin (mTHPC) and may be introduced intravenously. The illuminating may be performed between about two and about five days later. The tissue may be bladder tissue and may further comprise diagnosing cancer after illuminating based on the effects of the photoactive agent. The photoactive agent may comprise 5-aminolevulinic acid (ALA) configured for photodynamic therapy (PDT). The catheter may be inserted through an endoscope. The power may be sequentially varied at a frequency of about 45 Hz.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates a flexible circuit having light-emitting diodes (LEDs), according to an embodiment of the present disclosure.

FIG. 1B illustrates two of the flexible circuits of FIG. 1A arranged together around a shaft, according to an embodiment of the present disclosure.

FIG. 1C illustrates a catheter system including the flexible circuit of FIG. 1A, according to an embodiment of the present disclosure.

FIG. 1D illustrates the catheter system of FIG. 1C from another perspective.

FIG. 2A illustrates a catheter for activation of a photoactive agent, according to an embodiment of the present disclosure.

FIG. 2B illustrates a cross-section of the catheter of FIG. 2A.

FIG. 3 illustrates a catheter system for activation of a photoactive agent, according to an embodiment of the present disclosure.

FIG. 4 illustrates a catheter system for activation of a photoactive agent being used during a procedure, according to an embodiment of the present disclosure.

### DETAILED DESCRIPTION

The detailed description should be read with reference to the drawings, which are not necessarily to scale, depict illustrative embodiments, and are not intended to limit the scope of the invention.

As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

As used herein, the term “target tissue” refers to an unhealthy, diseased (i.e., cancerous, pre-cancerous, etc.) or otherwise undesirable portion of tissue that may be healthy or unhealthy. A target tissue may also include tissues that are suspected of being unhealthy or diseased, but which require verification of their disease status by biopsy.

A number of medical procedures including, e.g., along the digestive tract, urinary tract, or respiratory system, may deliver photoactive agents towards a target tissue. The photoactive agents may be light activated to provide therapy for or near the target tissue. For example, PDT is a cancer therapy based on the photochemical reaction between a light activatable molecule or photosensitizing agent. In the presence of such photosensitizing agents, light having a particular wavelength and molecular oxygen may form reactive oxygen species (ROS) that may damage nearby cells, inducing inflammatory and immune responses. Such photosensitizing agents may include talaporfin sodium for oesophageal cancer therapy or alpha lipoic acid for bladder cancer therapy, for example.

PDT may be ineffective for portions of target tissue at depths that make light penetration difficult or dangerous. For example, it may be problematic to activate photoactive agents at a depth of, e.g., greater than about 10 mm from a tissue surface. Precise wavelength illumination delivery, detection, and management may reduce complications with providing therapy at such depths.

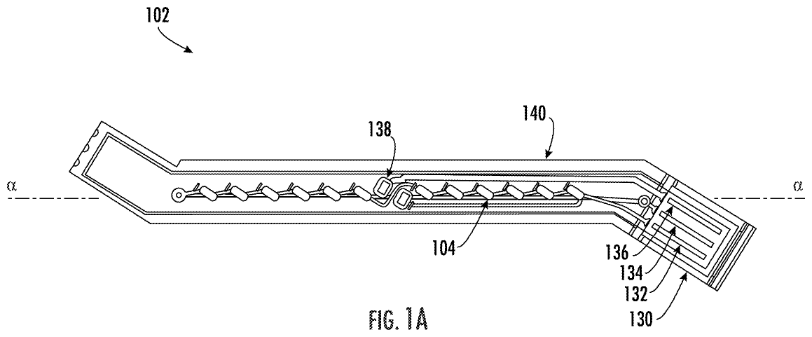

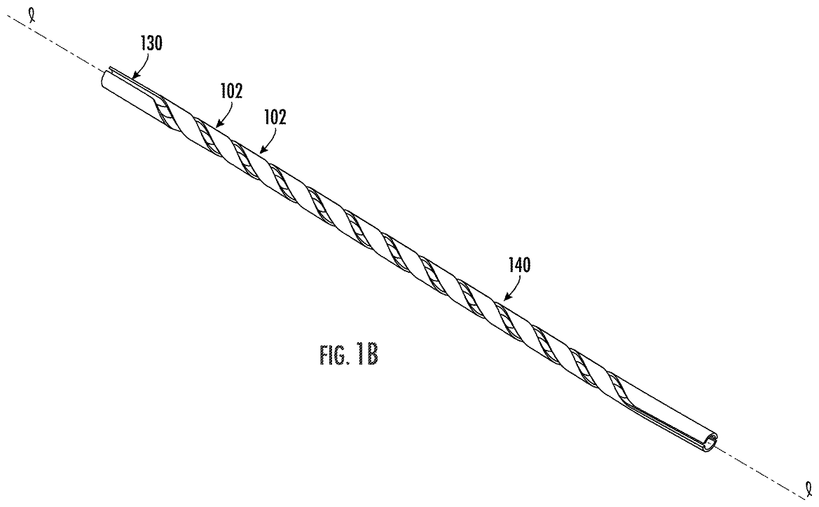

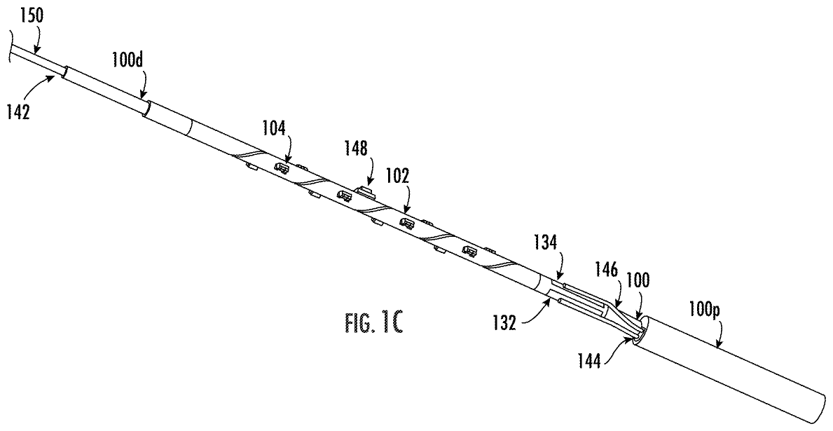

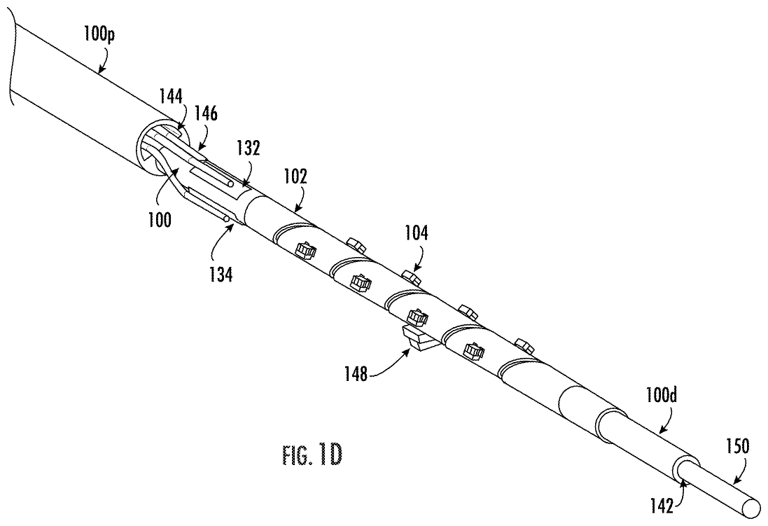

Referring to FIG. 1A, a flexible circuit 102 is illustrated according to an embodiment of the present disclosure. The circuit 102 includes 12 LED mounts 104 electrically coupled in series. Although 12 LEDs are illustrated, in various embodiments any number of LEDs may be employed, e.g., 1, 2, 3, 4, 5, 6, 8, 10, 15, 20, 30, 50, 100, etc. An end of the circuit 102 includes a connector 130 having lead contacts 132, 134, 136 for electrically communicating with components of the circuit 102. The connector 130 includes a supply lead contact 132, a return lead contact 134, and a sensing lead contact 136. The lead contacts 132, 134, 136 are configured for connection to a controller, power supply, and/or receiver for operating and feedback of the circuit 102. The end of the circuit 102 with the connector 130 extends at an oblique angle away from an axis a that extends parallel with a midportion 140 of the circuit 102. A pair of sensor mount points 138 (e.g., a pair of photodiodes) are disposed at a central location along the circuit 102 that may be used to detect one or more parameters of the system, e.g., wavelength, power, temperature, pH, or the like. Although a single pair of sensors mount points 138 are illustrated centrally along the circuit 102, in various embodiments, additional or alternative sensors mount points 138 may be employed and may be located at any location along the circuit 102. The circuit 102 may include heat sink material, e.g., copper, or the like, that may or may not also be used as part of electrical circuits.

Referring to FIG. 1B, two flexible circuits 102 of FIG. 1A are illustrated according with an embodiment of the present disclosure. The two flexible circuits 102 are arranged in a helical manner about a longitudinal axis € such that the flexible circuits 102 run parallel with each other. Although two flexible circuits 102 are illustrated, in various embodiments any number of flexible circuits 102 may be employed, e.g., 1, 3, 4, 5, 8, 10, 15, 20, 50, etc. The flexible circuits 102 may extend about a device (e.g., a catheter or the like) extending along the longitudinal axis f. Although the flexible circuits 102 are illustrated with a gap between them, in alternative embodiments, there may be no gap or the flexible circuits 102 may partially overlap. The ends of the flexible circuits 102 with the connector 130 are oriented toward the same end of the longitudinal axis l such that they can be electrically coupled to wires and/or one or more connectors. Because the ends of the flexible circuits 102 with the connector 130 extend away from the midportion 140 of the flexible circuit 102 at an oblique angle (i.e., as illustrated in FIG. 1A), the midportion 140 is able to be positioned helically about the longitudinal axis l without significant folds, creases, bends, etc. The two flexible circuits 102 may each be separately coupled to, e.g., a controller, such that a supply of power may be independently varied among one flexible circuit 102 and another flexible circuit 102. One or more flexible circuits 102 may be arranged having an outer diameter such that they may fit within a working channel of a scope, e.g., having an outer diameter of less than about 2.8 mm.

Referring to FIGS. 1C and 1D, a catheter system is illustrated including the flexible circuit 102 of FIG. 1A, according to an embodiment of the present disclosure. The system includes a catheter 100 that comprises an elongate shaft having a proximal portion 100p and a distal portion 100d. The proximal portion 100p has a larger outer diameter than an outer diameter of the distal portion 100d. A first lumen 142 extends along the catheter 100 and is accommodating a guidewire 150 therethrough, although other devices and/or fluids may additionally or alternatively extend through the first lumen 142. The flexible circuit 102 is disposed about the distal portion 100d of the catheter 100 in a helical manner. Although one flexible circuit 102 is illustrated in FIGS. 1C and 1D, it should be appreciated that any number of flexible circuits 102 may be employed, e.g., the two flexible circuits 102 as illustrated in the arrangement of FIG. 1B, or more. LEDs 104 are spaced along the first flexible circuit 102 such that they are substantially uniform in an array about the distal portion 100d. The proximal portion 100p of the catheter 100 includes a second lumen 144. Communications cables 146 (e.g., wires) are disposed within the second lumen 144 and may electrically couple the LEDs 104 with a control unit. The second lumen has a non-circular shape (e.g., oblong, c-shaped, crescent-shaped, or the like, but may also be circular) compared to the first lumen 142, which may better accommodate multiple communications cables 146 compared to the close-fit circular first lumen 142 accommodating the guidewire 150. Communications cables 146 may connect to each of the supply lead contact 132 and the return lead contact 134. A sensor 148 is disposed along the flexible circuit 102 at about a middle portion of the array of LEDs 104. A further communications cable 146 may connect to the sensing lead contact 136. The sensor 148 may be, e.g., a photodiode, a thermistor, a pH sensor, a pulse oximeter, or the like to provide feedback to a controller and/or a user of the region of the patient along the LEDs 104 before, during, and/or subsequent to operation.

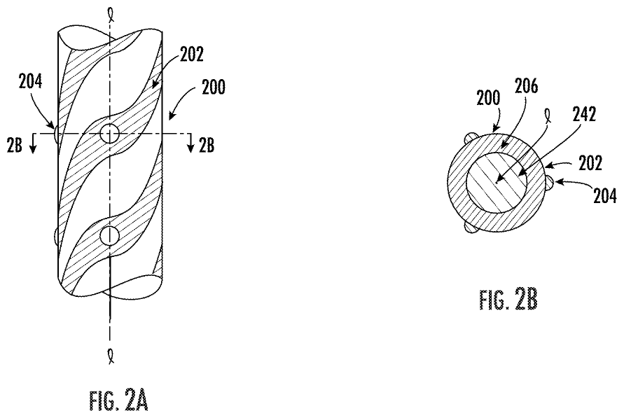

Referring to FIG. 2A, a catheter 200 for activation of a photoactive agent according to an embodiment of the disclosure is illustrated. The section of the catheter 200 illustrated is a distal portion of the catheter 200. Three circuits 202 are disposed helically about the distal portion of the catheter 200. Although three flexible circuits 202 are illustrated, in various embodiments any number of flexible circuits 202 may be employed, e.g., 1, 2, 4, 5, 8, 10, 15, 20, 50, etc. Multiple LEDs 204 are disposed along each of the circuits 202. The LEDs 204 amongst adjacent circuits 202 are aligned parallel with a longitudinal axis l of the catheter 200. The cross-section of the catheter 200 illustrated in FIG. 2B is taken normal to the longitudinal axis l at an LED 204 of each of the three circuits 202 such that an LED 204 of each circuit 202 is coincident with the cross-section 2B. A lumen 242 extends through the catheter 200. The lumen 242 may accommodate a guidewire and/or other devices and/or fluids. One or more communications cables electrically coupled independently to each of the circuits 202 may be disposed within the lumen 242 and/or extend through a wall 206 of the catheter to each of the circuits 202. Because each of the three circuits 202 are independently coupled electrically to a power source and/or a controller, each of the three circuits 202 are independently activatable.

In various embodiments herein, one or more LEDs or one or more sets of LEDs, e.g., along one or more circuits may be independently activated and/or powered. A treatment target location, target tissue, or adjacent tissue may undesirably be affected, e.g., heated by the one or more LEDs. To reduce or inhibit undesirable effects of LED emissions, one or more circuits may be activated or deactivated. Such activation and deactivation may be achieved by independently controlling the circuits. Circuits may be activated/deactivated sequentially (e.g., in a patterned fashion) such that LED emissions are uniformly (e.g., about or along a longitudinal axis) applied to a treatment area. For example, a helical circuit may be activated for a period at a power and sequentially may be deactivated or reduced in power while an adjacent helical circuit may be activated or increased in power. In this example, an effective LED emission from the collective circuits may be maintained without exceeding an undesirable emission from a particular LED or circuit (e.g., an overheated LED or oversaturation of light). Additionally, or in the alternative, LEDs amongst different circuits may have variable wavelengths. Such LED or circuit emissions may be monitored by one or more sensors along one or more of the circuits. Activating, deactivating, increasing power, or decreasing power may be performed manually or automatically by a controller. For example, a controller may oscillate power to LEDs of a circuit or amongst LEDs of different circuits in an oscillating manner such that continuous light is emitted during operation without LEDs overheating.

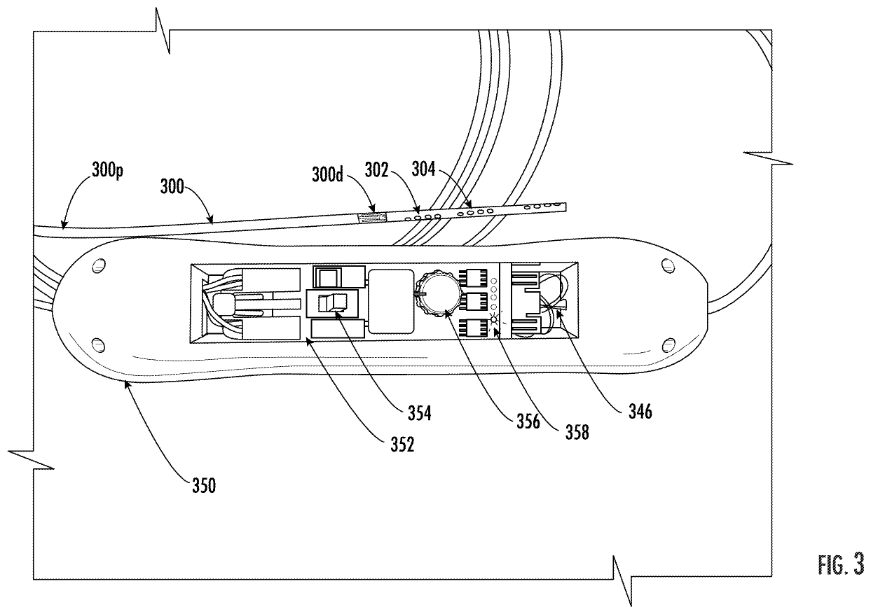

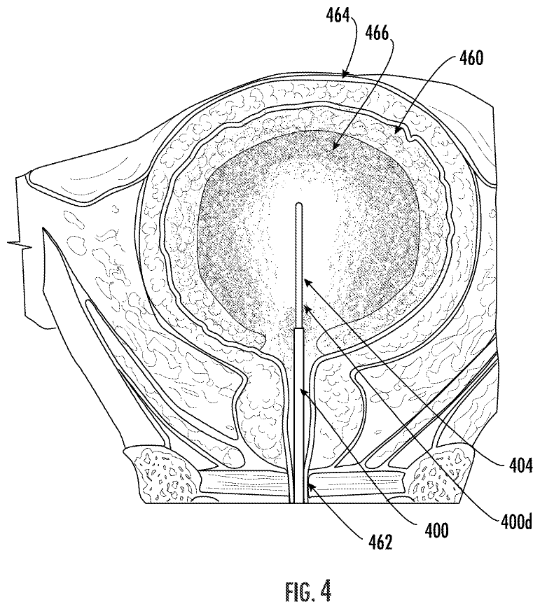

Referring to FIG. 3, a catheter system is illustrated according to an embodiment of the present disclosure. The system includes a catheter 300 that is an elongate shaft having a proximal portion 300p and a distal portion 300d. A circuit 302 is disposed along the distal end 300d. The circuit 302 includes twelve LEDs 304, although in various embodiments, any number of LEDs may be employed, e.g., 1, 2, 5, 10, 20, 50, etc. A control unit 350 is coupled to a proximal end of the catheter 300. The control unit 350 is ergonomically shaped to be handled by a user's hand(s). The control unit 350 contains a battery power supply (e.g., two 9V batteries within the control unit 350 but not illustrated) connected to a circuit board 352 operable by a switch 354. The circuit board 352 within the control unit includes an integrated circuit controller that is electrically coupled to the LEDs 304 by communications cables 346. The circuit controller is able to vary power provided to or otherwise adjust the intensity of the plurality of first LEDs 304 by adjusting an analog knob 356. Alternatively, the knob 356 may be configured for switching activation or adjusting oscillation rate or power level between multiple circuits 302 of LEDs 304. Indicator LEDs 358 may indicate to a user a state of the system, e.g., that the system is powered on or off, that portions or sets of the LEDs 304 are activated or unactivated, a system parameter such as heat, wavelength, duration of procedure, oscillation, or the like. The control unit 350 including battery power and user-interactive controls may be easier to manipulate and adjust the system compared to a system connected to stationary capital equipment. The battery power supply may be configured to detachably connect to a primary power supply for charging the battery power supply or may be removably replaced. Although a catheter 300 is illustrated in FIG. 3, it should be understood that any catheter described herein could be couplable to the control unit 350 and operate in a substantially similar fashion.

Referring to FIG. 4, a catheter system for activation of a photoactive agent 460 during a procedure is illustrated, according to an embodiment of the present disclosure. A catheter 400 is inserted through the male urethra 462 and into the bladder 464. The catheter 400 includes LEDs 404 disposed about a distal portion 400d of the catheter 400. The LEDs 404 are active, illuminating light 466 (that may or may not be on the visible spectrum). The light 466 is received by a photoactive agent 460 on or within the body lumen tissue of the bladder 464.

In various embodiments, an LED may be any shape, e.g., rectangular, oval, circular, oblong, a combination thereof, or the like. Additionally, or alternatively, a number of LEDs may be arranged in any shape, including a triangular shape (e.g., three LEDs angled 120 degrees relative to each adjacent LED) a square (e.g., four LEDs angled degrees relative to each adjacent LED), and/or may form an inline orientation with each LED facing a different direction. A light emitted from an LED may be emitted across a layer adjacent the LED that may be transparent or translucent. The layer may be a covering, such as an electrically insulating heat shrink or another substance, for example, a hardening or curable substance, such as an adhesive, that may be disposed along one or more portions of a circuit along a catheter. One or more LEDs may be arranged in multiple rows, e.g., two or more rows parallel or substantially parallel to a longitudinal axis and/or may be arranged circumferentially about the axis. LEDs may extend completely or partially about the circumference.

In various embodiments, LEDs, may extend along a portion of a length of a catheter. The LEDs may be arranged in various patterns along the elongate member, e.g., helically, axially, radially, circumferentially, linearly, intermittently spaced, randomly, at various densities along the length, or a combination of arrangements thereof, or the like, such that the LEDs may emit light along one or multiple radial angles from a longitudinal axis of the catheter. Light emitted from actuated LEDs may affect a photoactive agent and/or tissue (e.g., to perform tissue ablation). The LEDs may emit variable wavelengths of light that may result in various effects on photoactive agents and/or tissue, e.g., non-visible light (e.g., infrared or ultraviolet light) or visible light such as red, green, or the like such as between about 200 nm to about 700 nm. The LEDs may be actuated at varying frequencies via pre-programming or as manually controlled by a user. LEDs may be individually selectable and controllable as a single LED or in a series of more than a single LED. LEDs may be controllable by one or more parameters, such as density, location of the LEDS with respect to each other, anatomies, medical devices, size, shape, frequency of actuation, associated photoactive agents, intensity of actuation (e.g., using a current source, e.g., with pulse width modulation (PWM) operating at a desired frequency and/or duty cycle affecting LED intensity), duration of actuation, color, or any combination thereof.

In various embodiments, multiple sets of LEDs may be arranged along and/or about a catheter in various patterns. For example, a first set may be arranged along a first portion and a second set (and/or additional sets) may be arranged along a second portion opposite the first portion. For another example, a first set may be arranged helically about a catheter and a second set (and/or additional sets) may be arranged helically about the catheter adjacent the first set. Sets of LEDs may be independently activatable such that they may, e.g., flash (i.e., be powered on and off or powered and then reduced in power), in a pattern. For example, LEDs may flash at about 45 Hertz, with a duty cycle about 30% to about 75% and a current of approximately 30 mA. In various embodiments, LEDs may be chosen for a device depending on the procedure and/or photoactive agent(s) to be used.

In various embodiments, a device may include a plurality of wires, e.g., a positive wire connection to each LED anode terminal and a common cathode return terminal wire (either per LED or per set of LEDs), for conveying electrical energy from an electrical source (e.g., an electrical generator, a battery pack, or similar energy source) to provide control. Wires may also include a sensor wire. For example, wires may be contained in a lumen or a layer of a catheter and/or wires may extend along an outer surface of the catheter. The wires may also be insulated with, e.g., a biocompatible polymer.

In various embodiments, a distal end of a device may include a disperser in addition to or in place of one or more LEDs. A disperser may dampen and/or spread emitted light from one or more LEDs. In various embodiments, a plastic or a glass diffused light pipe may extend along the catheter. The catheter may include reflective surfaces along its length to advance light to a distal end of the catheter. For example, a light source may be coupled at a proximal end of the catheter.

In various embodiments, a controller may be electrically coupled to LEDs along a catheter. Electrical leads may be interleaved in electrical communication with the LEDs such that the LEDs may be independently actuated or independently activated in series of LEDs. The controller may be configured to sequentially actuate one or more of the LEDs at a time. The controller may be configured to sequentially actuate the LEDs such that only a single LED or multiple LEDs is/are actuated at once, e.g., LEDs along a first portion of the catheter and sequentially LEDs along a second portion of the catheter. The controller may be configured to actuate the LEDs at a higher frequency with reduced illumination time (e.g., a lower duty cycle having a lower percentage of time that the LEDs are illuminated) to reduce heat compared to LEDs that are actuated at a lower frequency with a longer illumination time (e.g., a higher duty cycle having a larger percentage of time that the LEDs are illuminated). The controller may be configured to actuate a portion of the LEDs at a first frequency and another portion of the LEDs at a second frequency, e.g., a distal portion of LEDs at a higher frequency than a proximal portion of LEDs. The LEDs may extend along the length of the elongate member for a distance that substantially aligns with an anatomy of a patient. The controller may be manually or automatically operated to actuate one or more LEDs or to switch between various patterns of operation. Such actuation of LEDs may be shortened to reduce heat. LEDs discussed herein and/or the activation time of such LEDs may have reduced heat about the system compared to other techniques and may activate a photoactive agent in less time than other techniques (e.g., compared to a bulb, laser, or the like).

In various embodiments throughout the disclosure, a method of activating a photoactive agent may include introducing the photoactive agent into or toward a tissue of a patient. A catheter having a first plurality of LEDs and a second plurality LEDs may be inserted into the patient towards the tissue. The photoactive agent may be illuminated by sequentially varying a power supplied to the first plurality of LEDs and the second plurality of LEDs. The tissue temperature may be monitored, and the power supplied may be varied based on the monitoring. The photoactive agent may be locally introduced into the tissue. The photoactive agent may be intravenously introduced into the patient. The tissue may be bladder tissue, pancreatic tissue, esophageal tissue, lung tissue, or the like. Light may be emitted from the first and/or second plurality of LEDs. The photoactive agent may comprise one of an anti-cancer compound and a photocurable, or a photocrosslinkable agent, or the like. The photoactive agent may comprise tetra(hydroxyphenyl)chlorin (mTHPC) and may be introduced intravenously. Illuminating may be performed between about two and about five days later. A photoactive agent could be introduced within a catheter including the LEDs and thereafter or simultaneously within the same procedure may be activatable. The tissue may be bladder tissue. Diagnosing cancer may be performed after illuminating based on the effects of the photoactive agent. The photoactive agent may comprise 5-aminolevulinic acid (ALA) configured for photodynamic therapy (PDT). Hexvix blue light cystoscopy (BLC) may be use as a diagnostic method for patients with bladder cancer. The catheter may be inserted through an endoscope. The power may be sequentially varied at a frequency of about 45 Hz.

It will be apparent to those skilled in the art that various modifications and variations can be made to the disclosed device without departing from the scope of the disclosure. For example, the configuration of devices for activation of a photoactive agent, may be altered to suit any medical therapy. It will be understood that the number and/or location of LEDs is not limited to the examples described herein. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A catheter system for activation of a photoactive agent, comprising:
a catheter comprising:
an elongate shaft comprising a proximal portion, a distal portion, and a first lumen extending along the elongate shaft;
a first flexible circuit disposed about the distal portion of the elongate shaft;
a plurality of first light-emitting diodes (LEDs) disposed along the first flexible circuit;
a second flexible circuit disposed about the distal portion of the elongate shaft; and
a plurality of second LEDs disposed along the second flexible circuit;
a control unit coupled to the proximal end of the elongate shaft;
a communications cable disposed within the catheter and electrically coupling the first and second LEDs with the control unit; and
wherein the control unit comprising a battery power supply and a controller comprising an integrated circuit electrically coupled to the plurality of first and second LEDs configured to vary power provided to the plurality of first and second LEDs;
wherein the controller is configured to vary power provided to the plurality of second LEDs independently of the first LEDs.

2. The catheter system of claim 1, wherein the first flexible circuit and the second flexible circuit are disposed about the distal end of the elongate shaft in a helical manner.

3. The catheter system of claim 1, wherein a cross-section of the catheter system taken normal to the first lumen at an LED of the first LEDS is coincident with an LED of the second LEDs.

4. The catheter system of claim 1, wherein the controller is configured to sequentially vary power between the plurality of first LEDs and the plurality of second LEDs.

5. The catheter system of claim 4, wherein the controller is configured to sequentially vary power to a frequency of about 45 Hz.

6. The catheter system of claim 1, wherein the first LEDs are configured to emit light at a first wavelength and the second LEDS are configured to emit light at a second wavelength different than the first wavelength.

7. The catheter system of claim 1, wherein at least one LED of the first LEDs is configured to emit a frequency of light different than a frequency of light emitted by another LED of the first LEDs.

8. The catheter system of claim 1, further comprising a photodiode disposed on the first flexible circuit configured to detect light emitted from the first LEDs.

9. The catheter system of claim 1, further comprising:
a second lumen extending through the proximal portion and the distal portion of the elongate shaft, the second lumen configured to accept at least one of a guidewire, the photoactive agent, and a fluid to be delivered into a patient;
wherein the first lumen extends along the proximal portion of the elongate shaft but not the distal portion of the elongate shaft.

10. A catheter for activation of a photoactive agent, comprising:
an elongate shaft comprising a proximal end, a distal end, and a first lumen therethrough;
a plurality of circuits disposed helically about the distal end of the elongate shaft;
a plurality of light-emitting diodes (LEDs) disposed along each of the plurality of circuits; and
a communications cable disposed within the first catheter lumen and electrically coupled independently to each of the plurality of circuits such that each of the plurality of circuits are independently activatable.

11. The catheter of claim 10, wherein a cross-section of the catheter taken normal to the lumen at an LED of the plurality of LEDS is coincident with an LED of each of the plurality of circuits.

12. The catheter of claim 10, wherein the LEDs of one circuit of the plurality of circuits are configured to emit light at a wavelength different than a wavelength of the LEDs of the remaining plurality of circuits.

13. The catheter of claim 10, further comprising a photodiode disposed on one of the plurality of circuits configured to detect light emitted from the plurality of LEDs.

14. The catheter of claim 10, wherein the communications cable is configured to sequentially transfer power independently among each of the plurality of circuits.

15. A method of activating a photoactive agent, comprising:
introducing the photoactive agent into a tissue of a patient;
inserting a catheter comprising a first plurality of light-emitting diodes (LEDs) and a second plurality LEDs into the patient towards the tissue; and
illuminating the photoactive agent by sequentially varying a power supplied to the first plurality of LEDs and the second plurality of LEDs.

16. The method of claim 15, further comprising monitoring tissue temperature and varying the power supplied based on the monitoring.

17. The method of claim 15, wherein the photoactive agent comprises one of an anti-cancer compound and a photocurable or a photocrosslinkable agent.

18. The method of claim 15, wherein the photoactive agent comprises tetra(hydroxyphenyl)chlorin (mTHPC) and is introduced intravenously, and wherein the illuminating is performed between about two and about five days later.

19. The method of claim 15, wherein the tissue is bladder tissue and further comprising diagnosing cancer after illuminating based on the effects of the photoactive agent.

20. The method of claim 15, wherein the photoactive agent comprises 5-aminolevulinic acid (ALA) configured for photodynamic therapy (PDT).
